(19) United States
(12) Patent Application Publication
(10) Pub. No.: US 2020/0093835 A1
(43) Pub. Date: Mar. 26, 2020

(54) METHODS OF TREATING VESTIBULAR SCHWANNOMA

(71) Applicant: Massachusetts Eye and Ear Infirmary, Boston, MA (US)

(72) Inventors: Konstantina Stankovic, Boston, MA (US); Jessica Elysse Sagers, Cambridge, MA (US)

(21) Appl. No.: 16/612,317

(22) PCT Filed: May 23, 2018
(86) PCT No.: PCT/US2018/034168

§ 371 Date: Nov. 8, 2019

Related U.S. Application Data
(60) Provisional application No. 62/511,116, filed on May 25, 2017.

(51) Int. Cl. A61K 31/567; A61K 9/00; A61P 35/00

(52) U.S. Cl. CPC A61K 31/567; A61K 9/0046; A61P 35/00; A61K 9/0053; A61K 9/0019

(57) ABSTRACT

Methods to reduce the proliferation of vestibular schwannoma (VS) cells and treat VS in a subject comprising administering a therapeutically effective amount of mifepristone.

### CLAIM OF PRIORITY

This application claims the benefit of U.S. Provisional Patent Application Ser. No. 62/511,116, filed on May 25, 2017. The entire contents of the foregoing are hereby incorporated by reference.

### FEDERALLY SPONSORED RESEARCH OR DEVELOPMENT

This invention was made with Government support under Grant Nos. DC015824 and DC00038 awarded by the National Institutes of Health, and Grant No. W81XWH-15-1-0472 awarded by the United States Department of Defense. The Government has certain rights in the invention.

### TECHNICAL FIELD

The present invention relates to methods to reduce the proliferation of vestibular schwannoma cells. The methods include administering mifepristone.

### BACKGROUND

Vestibular schwannoma (VS) is the fourth most common intracranial tumor and the most common tumor of the cerebellopontine angle, arising from neoplastic Schwann cells of the vestibular nerve. No drug is FDA-approved to treat VS. In 95% of patients, these tumors cause debilitating sensorineural hearing loss (SNHL) and tinnitus and often lead to dizziness and facial paralysis. Bilateral VSs are the hallmark of neurofibromatosis type 2 (NF2), an autosomal dominant disorder caused by inactivation or loss of both alleles of the NF2 gene. If left untreated, growing VSs can compress the brainstem and lead to death. Currently, patients with symptomatic or growing VSs can undergo surgical resection or radiotherapy, both procedures that can result in serious complications.

### SUMMARY

Bilateral VSs are the hallmark of neurofibromatosis type 2 (NF2), an autosomal dominant disorder caused by inactivation or loss of both alleles of the NF2 gene. Mutations in the NF2 gene are identified in 100% of NF2-associated VSs and 66% of sporadically arising VSs^{6,7}. Though mechanisms of VS-induced SNHL are multifactorial, with contributions from tumor size, localized or systemic infection, inflammation, and tumor-secreted factors^{8,9}, NF2-associated SNHL often correlates with VS size^{8,10}. This observation suggests that slowing or inhibiting VS growth may not only prolong a patient's time to surgical intervention, but also minimize or prevent associated SNHL, substantially improving quality of life.

Using publicly available omics data to probe relationships between genes, small molecules, and disease, the computational repositioning of existing drugs represents an appealing avenue for identifying potentially effective compounds, particularly for diseases with no FDA-approved pharmacotherapies. Here we present the first application of algorithm-based drug repositioning to neuro-otology, culminating in the computational repositioning and preclinical validation of mifepristone for human vestibular schwannoma (VS), a debilitating intracranial tumor. We applied ksRepo, an open-source computational drug repositioning platform³, to the largest meta-analysis of transcriptomic data from human VS patients, identifying eight promising drugs approved by the FDA with potential for repurposing in VS. Of these eight, we showed that mifepristone, a progesterone and glucocorticoid receptor antagonist, adversely affects the morphology, metabolic activity, and proliferation of HEI-193 human schwannoma cells, as well as that of primary human VS cells. Mifepristone treatment produces a more dramatic reduction in the metabolic activity of primary human VS cells than cells derived from patient meningiomas, while primary human Schwann cells remain unaffected.

Thus, provided herein are methods for reducing the proliferation of a vestibular schwannoma cell, wherein the method comprises contacting the vestibular schwannoma cell with an effective concentration of mifepristone. In some embodiments, the vestibular schwannoma cell is ex vivo; in some embodiments, the vestibular schwannoma cell is in a subject, e.g., a mammal, e.g., a human.

In some embodiments, the subject is or has been diagnosed as having vestibular schwannoma, e.g., using methods known in the art.

Also provided herein are methods for treating a subject having vestibular schwannoma. The methods include administering to the subject a therapeutically effective amount of mifepristone.

Further provided herein are methods for reducing the rate of vestibular schwannoma tumor growth in a subject that include administering to the subject a therapeutically effective amount of mifepristone.

In addition, provided herein are methods for inducing or increasing vestibular schwannoma cell death in a subject in need thereof that include administering to a subject a therapeutically effective amount of mifepristone.

In some embodiments of the methods described herein, the administration is local administration, e.g., by injection through the ear drum, or direct delivery into the inner ear.

In some embodiments of the methods described herein, the administration is systemic administration.

In some embodiments of the methods described herein, the systemic administration is oral, intravenous, intraarterial, nasal, intramuscular, subcutaneous, or intraperitoneal administration.

In some embodiments of the methods described herein, the subject has been diagnosed as having vestibular schwannoma.

In some embodiments of the methods described herein, the methods include a step of identifying or diagnosing a subject as having vestibular schwannoma.

The present methods can also be used in subjects having closely related schwannomas arising from other cranial nerves, such as schwannoma of the oculomotor, trigeminal, facial, hypoglossal, or vagal nerves within or outside of the parapharyngeal space and cutaneous schwannomas.

As used herein, the word “a” before a noun represents one or more of the particular noun. For example, the phrase “a vestibular schwannoma cell” represents “one or more vestibular schwannoma cells.”

The term “subject” means a vertebrate, including any member of the class mammalia, including humans, rats, mice, rabbits, sports or pet animals, such as horse (e.g., race horse) or dog (e.g., race dogs), and higher primates. In preferred embodiments, the subject is a human.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D. Computational repositioning of FDA-approved drugs using ksRepo. A, schematic depicting ksRepo workflow. B, largest meta-analysis to date of genome-wide expression in VS, comprising 80 tumors and yielding 1,335 commonly dysregulated genes, 405 of which were found to be significantly differentially expressed after Bonferroni correction for multiple hypothesis testing (p<0.05); analysis with ksRepo yields 8 drugs with high potential for repositioning in sporadic and NF2-associated VS. C, drug classes of repositionable candidates from ksRepo analysis. D, significant enrichment of anti-inflammatory, anti-neoplastic, and hormone-related drugs from all FDA-approved drugs after ksRepo analysis of genome-wide expression in VS (***: p<0.001).

FIGS. 2A-2I. Mifepristone adversely affects HEI-193 cells in culture. A, the metabolic viability of HEI-193 cells decreases with increasing concentration of mifepristone after 72 h in culture. Individual data points represent metabolic activity as percentage of vehicle-treated control for seven independent experiments, each performed in replicates of 3-5 wells per condition (MTT assay; vehicle-treated cells versus 35 μM mifepristone-treated cells, p=0.006; **: p<0.01). B, phase object confluence remains constant among cells treated for 72 hours with 35 μM mifepristone, while vehicle-treated cells exhibit normal proliferation patterns; cells quantified using nine replicate images per well per each treatment condition in duplicate. Error bars in the mifepristone-treated condition are smaller than the size of the symbol. C, 10× phase contrast images of vehicle-treated cells (top row) and cells treated with 35 μM mifepristone (bottom row) 6 h and 72 h post-treatment. D-F, the percentage of BrdU+ HEI-193 cells significantly declines after 72 h treatment with mifepristone (p=0.0007; ***: p<0.001): D, 25× epifluorescence image of cells treated with 0.1% DMSO vehicle, with BrdU in red and Hoechst stain in blue; E, cells treated with 35 μM mifepristone, where the white box encloses a single BrdU+ cell; F, quantification of five replicate experiments. G, activation of fluorescent caspase 3/7 detected via live-cell imaging over 100 h of mifepristone treatment reveals a slight but not significant elevation in caspase 3/7 activity between mifepristone-treated cells (70 μM and 35 μM) and vehicle-treated control cells; positive control is staurosporine (0.5 μM) and drug is applied 48 h after plating. Note that after 96 h in culture, normally proliferating vehicle-treated cells become overly confluent and begin to apoptose. H-I, phalloidin staining of mifepristone-treated cells reveals crumpled f-actin morphology after 72 hours: H, cells treated with 0.1% DMSO vehicle; I, cells treated with 35 μM mifepristone, where rhodamine phalloidin is red and Hoechst stain is blue. Center values in histograms are means; error bars are s.e.m.

FIGS. 3A-3B. Tumor size and NF2 mutation type show no relationship with mifepristone response. A, MRI scans of six VS patients whose primary tumor cells were treated with mifepristone after surgical resection (for scans of additional, non-sequenced tumors, see FIG. 5). White rectangles, VSs; white circles, second (smaller) VSs in NF2 patients, who presented with bilateral VSs. Scale bars, 20 mm. Scans for VS 1 and VS 4 were conducted without the use of contrast agent due to patient intolerance. B, schematic of NF2 gene (above) and resulting mRNA (below), describing mutation locus, mutation type, and resulting amino acid change for each tumor in A. Tumors 2 and 4 each contained two mutations in the NF2 gene, while for tumors 5 and 6, NF2 mutations were not found.

FIGS. 4A-4G. Mifepristone adversely affects primary human VS cells and human-derived arachnoid cells in culture, but leaves primary human Schwann cells unaffected. A, metabolic activity of primary VS cells declines with increasing concentrations of mifepristone; individual data points represent metabolic activity as percentage of vehicle-treated control for ten individual tumors, performed in replicates of 3-5 wells per condition (vehicle-treated cells versus 35 μM mifepristone-treated cells, p=0.002; **: p<0.01). B, quantification of the significant decline in BrdU incorporation observed in primary VS cells treated with 35 μM mifepristone (p=0.0002; ***: p<0.001). C-E, live cell fluorescence microscopy reveals a significant increase in cytotoxicity of VS cells under mifepristone treatment; representative data from a single tumor, quantified from nine replicate images per treated well, performed in quadruplicate for each treatment condition: c, primary human VS cells imaged at 10× after treatment with 35 μM mifepristone for 72 hours, where cytotoxicity is indicated via green fluorescent signal; D, vehicle-treated control cells; E, quantification of cytotoxicity, reported as number of green objects per well after thresholding to exclude small cellular debris. F, mifepristone reduces the metabolic viability of schwannoma cells more significantly than that of AC-CRISPR NF2(−/−) and AC-CRISPR NF2(+/+) human arachnoid cells. G, mifepristone does not adversely affect the metabolic viability of primary human Schwann cells in culture (p=0.23). Center values in histograms are means; error bars are s.e.m.

FIG. 5. Ingenuity Pathway Analysis (Qiagen) highlights mifepristone as a significant upstream regulator of predicted regulatory networks generated after the analysis of all genes in the 80-tumor meta-analysis that were identified as significantly differentially regulated after Bonferroni correction for multiple hypothesis testing (p=4.26*10−5). Dotted lines, theorized relationships; solid lines, known relationships.

FIG. 6. Enzyme-linked immunosorbent assay (ELISA) on conditioned cell culture medium collected from HEI-193 cells treated with 35 μM mifepristone and 0.1% DMSO vehicle (n=6 from mifepristone-treated cells, 2 from vehicle-treated cells). Mifepristone-treated cells showed an increase in progesterone in culture medium, suggesting that the drug is effectively competing with progesterone for receptor binding (two-tailed unpaired T test, p=0.08).

FIGS. 7A-7H. Flow cytometry for annexin V/propidium iodide staining and terminal deoxynucleotidyl transferase dUTP nick-end labeling (TUNEL) assay reveal no significant differences in the apoptotic cell fraction or phase of cell cycle in mifepristone-treated cells versus vehicle-treated controls. A, Annexin V and propidium iodide labeling of HEI-193 cells treated with 35 μM mifepristone for 72 h reveals a slight but not significant increase in early apoptotic cells as compared to vehicle-treated controls (quantified in C). B, cell cycle analysis exhibits no significant differences in phase of cell cycle after 72 h mifepristone treatment (quantified in D). E-H, TUNEL assay reveals no statistically significant difference between the number of TUNELpositive cells in the mifepristone-treated and vehicle-treated conditions (representative results; experiment repeated three times in duplicate, quantifying three fields of view per treatment condition): E, DNAse-treated positive control; F, TUNEL stain on cells treated with 0.1% DMSO vehicle; G, TUNEL stain on cells treated with M mifepristone for 72 h.; green, TUNEL; blue, Hoechst stain; red, rhodamine phalloidin. H, quantification of TUNEL assay (n=3).

FIG. 8. Four additional MRI scans of VS patients whose primary tumor cells were treated with mifepristone after surgical resection. White rectangles, VSs; scale bars, 20 mm.

FIGS. 9A-9H. Results of metabolic activity (MTT), proliferation (BrdU incorporation), and cell death (terminal dUTP nick end labeling, TUNEL) assays performed on HEI-193 and primary VS cells treated for 72 h with other drugs recommended by ksRepo. A, treatment with 25 μM prednisolone produces a modest effect on the metabolic activity of primary VS cells in culture; B, treatment with 25 M prednisolone produces no effect on the proliferation of primary VS cells in culture; C, treatment with 25 μM prednisolone produces a 15.85% elevation in cell death among primary VS cells when compared to vehicle-treated controls; D, treatment with 25 μM prednisolone produces no significant effect on HEI-193 cell metabolic activity; E, treatment with 27 μM methylprednisolone produces no significant effect on HEI-193 cell metabolic activity; F, treatment with 25, 50, and 100 μM succimer produces no significant effect on HEI-193 cell metabolic activity; G, treatment with 25, 50, 100, and 300 μM gold sodium thiomalate produces a significant effect on HEI-193 cell metabolic activity, but only at clinically unreasonable concentrations; H, treatment with 5, 10, and 15 μM adenosine monophosphate produces no significant effect on HEI-193 cell metabolic activity.

### DETAILED DESCRIPTION

Vestibular schwannomas (VSs), the most common tumors of the cerebellopontine angle, can cause substantial morbidity. There is a clinical need to develop pharmacotherapies against VS as current treatments carry significant risks. Described herein are specific pathways involved in the pathobiology of neoplastic VS growth and VS-associated SNHL and therapeutic targets that regulate neoplastic VS growth and VS-induced SNHL.

Clinical Features and Incidence of Vestibular Schwannomas (VSs)

Neoplastic Schwann cells (SCs) of the vestibular nerve lead to VSs, the fourth most common intracranial tumors. VSs, although benign in nature, can lead to various symptoms due to their crucial location within the internal auditory canal that houses the vestibulocochlear and facial nerves (Tew & McMohan, 2013). Ninety-five percent of VS patients suffer from sensorineural hearing loss (SNHL), with a smaller percentage suffering from vestibular dysfunction and facial nerve paralysis (Matthies & Samii, 1997). Further, due to their expansion into the cerebellopontine angle, VSs can lead to brainstem compression and death as the tumors grow larger (Charabi et al., 2000).

To alleviate this tumor burden, patients can undergo surgical resection or stereotactic radiotherapy. Surgical resection entails full or partial removal of the tumor via craniotomy and carries substantial risks, including SNHL, vestibular dysfunction, facial nerve paralysis, cerebrospinal fluid leaks and meningitis (Sughrue et al., 2011a; Mahboubi et al., 2014). Stereotactic radiotherapy entails delivering a radiation dose to the tumor and also carries substantial risks such as further exacerbation of the SNHL, vestibular dysfunction and malignant transformation of the tumor (Demetriades et al., 2010; Collens et al., 2011). Patients with non-growing or asymptomatic VSs can undergo conservative management and follow the tumor's progression through serial magnetic resonance imaging (MRI), but due to the lack of biomarkers for VS growth and associated symptoms, it can be a risky approach (Thakur et al., 2012). Reliable biomarkers and effective drug therapies would greatly advance health care for VS patients. In this disclosure, with an eye towards identifying effective biomarkers and pharmacotherapies, several pathobiological pathways in VS growth and VS associated SNHL were investigated.

Clinical incidence of VS has been approximately 19 per million per year (Stangerup & Cayé-Thomasen, 2012). The first VS and associated SNHL were described in 1830 by Sir Charles Bell and incidence rates have increased considerably over time, partially attributed to the advent of imaging. Although cell phone radiation-induced neoplastic transformation has been postulated, most studies investigating correlation of cell phone use with VS incidence show negative findings (Pettersson et al., 2014). Interestingly, histologic incidence for VS is approximately 1 per 500, as assessed through MRIs conducted on a group of 2000 subjects from the general population (Vemooij et al., 2007). Further, the vestibular nerve serves as a predilection site for schwannomas, with 57% of schwannomas occurring on this nerve (Propp et al., 2006). These unusually high incidence rates suggest an intriguing biology of the vestibular nerve and VS.

Methods of Treatment

The methods described herein can be used to treat subjects with VS, e.g., subjects who have been diagnosed with VS, or having closely related schwannomas arising from other cranial nerves, such as schwannoma of the oculomotor, trigeminal, facial, hypoglossal, or vagal nerves within or outside of the parapharyngeal space and cutaneous schwannomas. The methods include administering a therapeutically effective concentration of mifepristone. Mifepristone (RU486), 11β-(4-dimethylaminophenyl)-17β-hydroxy-17α-(1-propynyl)-estra-4,9-dien-3-one, is a progesterone and glucocorticoid receptor antagonist currently approved by the FDA for use in medical abortion. This steroid analog is able to cross the blood-brain barrier¹² and has been shown in human clinical trials to provide palliative benefits to patients with other intracranial tumors, such as glioblastoma multiforme¹² and meningioma^{13,14}. In vitro, mifepristone produces antiproliferative effects on cervical^{15,16}, breast^{17,18}, endometriall^{9,20}, ovarian^{21,22}, gastric²³, bile duct²⁴, and prostate cancer cells^{25,26}, regardless of progesterone receptor expression²⁷. In human trials, mifepristone administration has been documented to significantly improve quality of life for patients suffering from advanced thymic, renal, colon, leukemic, and pancreatic cancers^{28,29}. Long-term administration of oral mifepristone is well tolerated by adults and carries only a mild toxicity profile¹³.

In some embodiments, subjects treated with the present methods do not have, or have not been diagnosed with, a neurofibroma; in some embodiments, they have (or have been diagnosed with) a multiple schwannoma disorder, e.g., neurofibromatosis type 2, schwannomatosis, or Carney complex (See, e.g., Rodriguez et al., Acta neuropathologica. 2012; 123(3):295-319). In some embodiments, subjects treated with the present methods have bilateral VSs (and not neurofibromas), and may have neurofibromatosis type 2 (NF2).

There is no consensus about whether VS tumors even express PR. In 2006, an immunohistochemical study of 100 VSs found that no tumors expressed the PR (Jasiwal et al., Journal of Negative Results in Biomedicine. 2009; 8:9). In 2008, a similar immunohistochemical study of 59 tumors found that all tumors expressed the PR (Caifer et al., J Laryngol Otol. 2008 February; 122(2): 125-7). The same year, Dalgorf et al. showed that nine of nine tested VSs were “unequivocally negative” for PR (Dalgorf et al., Skull Base. 2008; 18(6):377-384). Later that year, Patel et al. claimed that 16 of 23 sporadic VSs upregulated PR messenger RNA (mRNA) expression, but that NF2-associated VSs significantly downregulated PR mRNA expression (Patel et al., The Laryngoscope. 2008; 118(8):1458-1463). Therefore, with no clear consensus in the field regarding PR expression in VS, McLaughlin et al.'s claim cannot be considered applicable to this tumor. In our own meta-analysis comprising 80 VSs, PR mRNA was concordantly downregulated (false discovery rate p=0.01). When testing mifepristone on primary human VS cells, we observed no correlation between response to mifepristone and PR expression. In some embodiments, the subject does not have a progesterone receptor (PR)-expressing VS; in these embodiments, the methods can include determining whether the VS expresses PR (see, e.g., WO2004010928), and excluding those that do express PR in the schwannoma cells.

In some embodiments, the methods include determining that the subject is not pregnant or not likely to become pregnant.

Pharmaceutical Compositions

The methods described herein can include administration of mifepristone as an active agent in a pharmaceutical composition. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

The present pharmaceutical compositions are formulated to be compatible with the intended route of administration.

In some embodiments, the compositions are delivered systemically, e.g., by oral, parenteral, e.g., intravenous, intradermal, or subcutaneous administration.

In some embodiments, the compositions are administered by local administration to the vestibular schwannoma, e.g., by application of a liquid, foam, or gel formulation to the round window membrane. Application to the round window membrane can be accomplished using methods known in the art, e.g., intra-tympanic injection of a liquid, foam, or gel formulation or by direct delivery into the inner ear fluids, e.g., using a microfluidic device such as an implantable pump.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the therapeutic compounds are prepared with carriers that will protect the therapeutic compounds against rapid elimination from the body, such as a controlled release formulation, including implants and microencapsulated delivery systems. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Nanoparticles, e.g., poly lactic/glycolic acid (PLGA) nanoparticles (see Tamura et al., Laryngoscope. 2005 November; 115(11):2000-5; Ge et al., Otolaryngol Head Neck Surg. 2007 October; 137(4):619-23; Horie et al., Laryngoscope. 2010 February; 120(2):377-83; Sakamoto et al., Acta Otolaryngol Suppl. 2010 November; (563):101-4) can also be used.

In some embodiments, the carrier comprises a polymer, e.g., a hydrogel, that increases retention of the compound on the round window and provides local and sustained release of the active ingredient. Such polymers and hydrogels are known in the art, see, e.g., Paulson et al., Laryngoscope. 2008 April; 118(4):706-11 (describing a chitosan-glycerophosphate (CGP)-hydrogel based drug delivery system); other carriers can include thermo-reversible triblock copolymer poloxamer 407 (see, e.g., Wang et al., Audiol Neurootol. 2009; 14(6):393-401. Epub 2009 Nov. 16, and Wang et al., Laryngoscope. 2011 February; 121(2):385-91); poloxamer-based hydrogels such as the one used in OTO-104 (see, e.g., GB2459910; Wang et al., Audiol Neurotol 2009; 14:393-401; and Piu et al., Otol Neurotol. 2011 January; 32(1): 171-9); Pluronic F-127 (see, e.g., Escobar-Chavez et al., J Pharm Pharm Sci. 2006; 9(3):339-5); Pluronic F68, F88, or F108; polyoxyethylene-polyoxypropylene triblock copolymer (e.g., a polymer composed of polyoxypropylene and polyoxyethylene, of general formula E106 P70 E106; see GB2459910, US20110319377 and US20100273864); MPEG-PCL diblock copolymers (Hyun et al., Biomacromolecules. 2007 April; 8(4):1093-100. Epub 2007 Feb. 28); hyaluronic acid hydrogels (Borden et al., Audiol Neurootol. 2011; 16(1):1-11); foams, e.g., as described in WO2009132050A9, WO2011049958A2, WO2015031393A1, or WO2010048095A2; gelfoam cubes (see, e.g., Havenith et al., Hearing Research, February 2011; 272(1-2):168-177); and gelatin hydrogels (see, e.g., Inaoka et al., Acta Otolaryngol. 2009 April; 129(4):453-7); other biodegradable, biocompatible polymers can be used, such as ethylene vinyl acetate, polyanhydrides, polyglycolic acid, collagen, polyorthoesters, and polylactic acid. Tunable self-assembling hydrogels made from natural amino acids L and D can also be used, e.g., as described in Hauser et al e.g. Ac-LD6-COOH (L) e.g. Biotechnol Adv. 2012 May-June; 30(3):593-603. Such formulations can be prepared using standard techniques, or obtained commercially, e.g., from Alza Corporation and Nova Pharmaceuticals, Inc. In some embodiments, the composition (e.g., in foam or gel form) is applied to the tympanic membrane, e.g., as described in WO2009132050A9, WO2011049958A2, WO2015031393A1, or WO2010048095A2.

The pharmaceutical compositions can be included in a container, pack, or dispenser together with instructions for administration.

Dosage

An “effective amount” is an amount sufficient to effect beneficial or desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. In some embodiments, e.g., in subjects exposed to prolonged or repeated exposures to noise, e.g., normal noises such as are associated with activities of daily life (such as lawnmowers, trucks, motorcycles, airplanes, music (e.g., from personal listening devices), sporting events, etc.), or loud noises, e.g., at concert venues, airports, and construction areas, that can cause inner ear damage and subsequent hearing loss; e.g., subjects who are subjected to high levels of environmental noise, e.g., in the home or workplace, can be treated with repeated, e.g., periodic, doses of the pharmaceutical compositions, e.g., to prevent (reduce the risk of) or delay progression or hearing loss.

Dosage, toxicity and therapeutic efficacy of the therapeutic compounds can be determined by standard pharmaceutical procedures, e.g., in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. For example, samples of the perilymph or endolymph can be obtained to evaluate pharmacokinetics and approximate an effective dosage, e.g., in animal models, e.g., after administration to the round window. The dosage of such compounds lies preferably within a range of concentrations that include the ED50 with little or no toxicity. The dosage may vary within this range depending upon the dosage form employed and the route of administration utilized. For any compound used in the method of the invention, the therapeutically effective dose can be estimated from cell culture assays, and/or a dose may be formulated in animal models; alternatively, for those compounds that have been previously used in humans, clinically desirable concentrations can be used as a starting point. Such information can be used to more accurately determine useful doses in humans.

In some embodiments, the dose is about 100-300 mg/day, e.g., about 200 mg/day, delivered orally.

### EXAMPLES

The invention is further described in the following examples, which do not limit the scope of the invention described in the claims.

Methods

The following materials and methods were used in the Examples below.

GEO Dataset Processing

The GEO dataset used in this study is GSE39645, an Affymetrix Human Gene 1.0 ST chip-based gene expression study of VS which contained data from 28 patients with sporadic VS, 3 patients with NF2-associated VS, and 8 control nerve samples⁴¹. Data for GSE39645 was accessed through the NCBI GEO portal and analyzed using the integrated GEO2R tool⁴². As input for GEO2R, we classified each sample within a GEO series as either normal tissue or VS tissue. The GEO2R analysis was performed on both the full dataset (sporadic and NF2 combined), and a subset of samples containing only NF2-syndromic schwannomas. GEO2R provides a list of probes and corresponding gene symbols ranked according to their degree of differential expression (as calculated using the limma package in R⁴³), and includes p-values and t-statistics for differential expression. Following GEO2R analysis, all results were imported into R⁴⁴ and probe-level differential expression was consolidated to gene-level differential expression using a custom pipeline: t-statistic values were converted to Cohen's d statistic values and standard error values⁴⁵. Resulting values were combined by gene using a fixed effects meta-analysis (as implemented in the meta.summaries function from the rmeta package in R⁴⁶. Probes without gene annotations were removed from gene-level consolidation. Following consolidation⁴⁷, significantly differentially expressed genes were taken to be those with a Bonferroni-corrected significance of less than 0.05.

Additional VS Dataset Processing

Raw Affymetrix Human Genome U219 gene expression data (.CEL files) for 36 patients with sporadic VS, 13 patients with NF2 syndrome-associated VS, and 7 control nerves were generously donated by Agnihotri et al.⁶. CEL files were loaded into R using the justRMA function from the affy package in R⁴⁸. justRMA is an automated tool that both performs normalization using the Robust Multi-Array Average method⁴⁹ and also automatically annotates all probes in the normalized dataset using the Org.Hs.eg.db annotation database package⁵⁰. Normalization was performed on the full dataset and the NF2-associated schwannomas, as above. Mirroring the GEO2R analysis, each normalized dataset was analyzed using limma and consolidated to gene-level differential expression using the custom pipeline described above. As above, significantly differentially expressed genes were taken to be those with a Bonferroni-corrected significance of less than 0.05.

Meta-Analysis of 80 VS Samples and ksRepo Prediction

To robustly determine differential expression between VS and normal tissues, gene-level data from GSE39645 and Agnihotri et al.⁶ were meta-analyzed by first removing genes that were not measured in both the Affymetrix Human Gene 1.0 ST chip and the Affymetrix Human Genome U219 chip, and subsequently combining Cohen's d and standard error values using a fixed-effects meta-analysis (again using meta.summaries). Meta-analysis was performed for the full GSE39645 and Agnihotri datasets, as well as for NF2-associated tumors exclusively. Following meta-analysis, the remaining genes were ranked according to their meta-analytic p-values to generate a gene list for further analysis using ksRepo (package available for download at github.com/adam-sam-brown/ksRepo, and described in Brown et al (2016)³. ksRepo is a gene-based drug repositioning method that uses a modified Kolmogorov-Smimov (KS) statistic to identify promising drug repositioning opportunities. ksRepo requires a database of compound-gene interactions, which are compared with the ranked meta-analytic gene lists from above. For this analysis, the ksRepo built-in Comparative Toxicogenomics Database (CTD) dataset was selected. The CTD provides a curated resource that links small chemical entities to genes (e.g., gene or protein expression influences) from the scientific literature on numerous model organisms and humans¹¹. ksRepo contains a subset of the CTD, containing human-derived interactions between 1,268 unique drugs and 18,041 unique human genes. Drugs in the CTD subset were chosen based on case-insensitive matches between CTD names and names/synonyms for FDA-approved drugs downloaded from DrugBank⁵¹. The ksRepo output provides both the resampled p-value and FDR value. For the full dataset ksRepo analysis and the NF2-only ksRepo analysis, significant compounds were those for which the FDR was less than 0.05.

Human Specimen Collection and Primary Cell Culture

Surgical VS and GAN specimens were collected and processed according to protocols approved by the Human Studies Committee of Massachusetts General Hospital and Massachusetts Eye and Ear (Board Reference #14-148H). Written informed consent was obtained from all subjects prior to inclusion in this study and all procedures were conducted in accordance with the Helsinki Declaration of 1975. Detailed methods for human surgical specimen collection, processing, and culture are previously published³⁸. VS specimens were harvested from patients undergoing surgical tumor resection, and GAN specimens from healthy patients undergoing benign parotidectomy or neck dissection surgery, during which the GAN is routinely sacrificed. Patients who had received radiation therapy prior to surgery were excluded.

Briefly, after surgical resection, VS or GAN tissue was immediately placed in saline solution and transported to the laboratory on ice. Specimens were rinsed with Hank's Balanced Salt Solution (HBSS, ThermoFisher Scientific), dissected to remove burned tissue and blood vessels, and separated for RNA preservation (RNALater, ThermoFisher Scientific) or primary cell culture. After enzymatic dissolution (collagenase type I, 160 U/mL; hyaluronidase type I-S, 250 U/mL) and trituration with an 18-gauge needle, primary cell culture suspensions were plated on 12 mm coverslips pre-coated with poly-D-lysine and laminin (Neuvitro) and grown in Dulbecco's Modified Eagle's Medium (DMEM) and F12-containing medium (ThermoFisher Scientific) consisting of 44.5% DMEM, 44.5% F12 nutrient mixture, 10% fetal bovine serum (ThermoFisher Scientific), and 1% of a mixture of penicillin and streptomycin (ThermoFisher Scientific). VS and GAN cultures were incubated at 37 degrees Celsius with 5% carbon dioxide, and culture medium was changed every three days. All downstream procedures were performed on primary cell cultures or collected culture medium at two weeks of age in culture to ensure maximal Schwann or schwannoma cell purity³⁸.

HEI-193 and Arachnoid Cell Culture

HEI-193 cells are derived from a patient with sporadic bilateral vestibular schwannomas and a history of meningioma; these cells express a splice variant of the merlin protein (encoded by the NF2 gene), but neither typical isoform⁵². HEI-193 cells were cultured in DMEM/F12-containing medium with 10% fetal bovine serum and 1% penicillin and streptomycin mix as described above. Immortalized NF2-null and NF2-expressing arachnoid AC-CRISPR cell lines derived from primary human autopsy specimens were obtained via generous gift from Dr. Vijaya Ramesh at Massachusetts General Hospital⁴⁰. NF2-null and NF2-expressing arachnoid cells were cultured in DMEM with 15% fetal bovine serum and 1% penicillin and streptomycin mix. All cell lines were maintained in an incubator at 37 degrees Celsius with 5% carbon dioxide and treated with drugs 24-36 hours after seeding at between 15,000-25,000 cells per well in 24-well plates. Phase contrast photos of healthy and drug-treated cultures were taken at 10× magnification on an IncuCyte S3 instrument (Essen Bio).

Drug Preparation and Treatment

Primary VS and GAN cultures were treated with mifepristone (Sigma Aldrich, lot # WXBC0031V) and progesterone (Sigma Aldrich, lot # SLBQ9723V). Fifteen, 25, 35, and 70 μM mifepristone, and 35 μM progesterone were prepared by suspending the appropriate amount of drug (in powder form) in dimethyl sulfoxide (DMSO). The resulting drug suspension was diluted in culture medium to the concentration of interest, and drug-containing medium was applied to primary VS, GAN, and HEI-193 cells such that the amount of DMSO applied to cells in culture did not exceed 0.1% (24-well plate, 1 mL medium per well). Cultures were incubated with drug-containing medium or 0.1% DMSO vehicle for 72 hours and then processed for downstream applications.

Proliferation Assay

5′bromo-2′-deoxyuridine (BrdU) was added to label proliferating cells in culture 2 hours before fixation in 4% formalin (paraformaldehyde). Cell membranes were permeabilized with 10 minutes of incubation in 1% Triton X-100 and nuclear membranes with 20 minutes in 2N hydrochloric acid (HCl). Cells were blocked in 5% normal horse serum (NHS) and 1% Triton X-100 and incubated with a primary antibody against BrdU (# OBT0030G, AbD Serotec) overnight, followed by incubation with fluorescent anti-rat immunoglobulin G (AlexaFluor, Life Technologies). Cells were stained with Hoechst 33342 (Invitrogen) and phalloidin/f-actin (ThermoFisher Scientific) and coverslips mounted on slides with VectaShield (Vector Laboratories). The ratio of BrdU-positive to Hoechst-positive nuclei was determined by sampling three random fields of view using a Leica epifluorescence microscope. Manual counts were performed by J.E.S., who was blinded to treatment conditions by receiving and quantifying image files labeled only with arbitrary numbers and presented in random order.

Metabolic Activity Assay

The metabolic activity of primary VS and HEI-193 cells was assessed using the colorimetric 3-(4,5-dimethylthiazol-2-yl)-2,5-diphenyltetrazolium bromide (MTT) assay (Life Technologies). After 72-hour incubation of cells in a 24-well plate with medium containing drug or vehicle, culture medium was replaced with 362 μL of colorless DMEM and 38 μL of 12 mM MTT in 3-6 random wells. Cells were incubated for an additional 4 hours. The resulting formazan crystals were dissolved in 380 μL of a solution of 100 mg/mL sodium dodecyl sulfate in 0.01 M HCl and incubated for another 4 hours. Optical density (OD) at 570 nm of each well was detected using a spectrophotometer. The average OD value of cells exposed to vehicle (0.1% DMSO) was set to 100% and used to normalize OD values of cells treated with drugs; metabolic activity was then reported as percent change. Statistical testing was performed on raw OD values (see Statistical Methods).

Flow Cytometry

Apoptotic cell death was assessed using an Annexin V/propidium iodide (PI) staining kit (Miltenyi Biotec). Briefly, HEI-193 cells were seeded into T25 flasks and treated with mifepristone or DMSO vehicle in culture medium for 24 hours as described above. Adherent cells were collected by trypsinization, and non-adherent (floating) cells were collected from culture medium. Cells were centrifuged, washed in PBS, and incubated in 1× annexin binding buffer, annexin V-fluorescein isothiocyanate (FITC), and propidium iodide (PI) according to the manufacturer's recommendations. Stained cells were immediately analyzed using a Cytomics FC500 flow cytometer. Data were analyzed using CXP Analysis software (version 2.2, Coulter).

Cell Cycle Analysis

Harvested HEI-193 cells were washed in PBS and fixed in cold 70% ethanol at −20° C. for 18-72 h. Before staining with propidium iodide (PI), cells were centrifuged and washed again in cold PBS. 2×10⁶ or fewer cells were incubated with 500 μl staining solution [0.1% Triton X-100 (Sigma), 2 mg/ml RNase A (Qiagen), and 1 μg/ml PI (Miltenyi Biotec) in PBS] for 15 minutes at 37° C. In order to exclude DNAse activity, RNase A was boiled for 5 minutes and cooled down before its addition to staining solution. Cells were analyzed on a Cytomics FC500 flow cytometer using CXP Analysis software (version 2.2, Coulter).

Enzyme-Linked Immunosorbent Assay

Cell-conditioned medium was collected from mifepristone-treated and untreated HEI-193 cells after 72-hour incubation with the drug. Enzyme-linked immunosorbent assay (ELISA) was performed on each sample in triplicate to assess the quantity of progesterone in cell-conditioned medium, according to the manufacturer's protocol (Enzo Life Science, # ADI-900-011). Data were analyzed using GraphPad Prism 7 software licensed through Harvard Medical School.

Cytotoxicity and Cell Confluence Assays

Cytotoxicity and cell confluence were measured using live-cell, time-lapse phase contrast and fluorescence imaging acquired at 10× by an IncuCyte S3 instrument (Essen Bioscience). Cytotoxicity was measured by incorporation of IncuCyte Cytotox Reagent (Essen Bioscience), applied according to manufacturer's instructions; the reagent fluoresces when it binds DNA after compromise of membrane integrity. Nine images were acquired per well every 2 hours for 72 hours, and cytotoxicity at each time point is reported as number of fluorescent objects per well, after thresholding to avoid the inclusion of small cellular debris. Phase object confluence was measured using time-lapse phase contrast imaging acquired at 10× by the same instrument, analyzing 9 images per well every 2 hours for 72 hours, and is reported as percent confluence per square millimeter.

Ingenuity Pathway Analysis

Ingenuity Pathway Analysis software (Qiagen) was used to perform standard Core Analysis on all genes in our meta-analysis that reached significance (p<0.05) after Bonferroni correction for multiple hypothesis testing. Relevant upstream regulators for the resulting networks were identified and analyzed using published Ingenuity Pathway Analysis protocols (Qiagen).

gDNA Extraction

Genomic DNA (gDNA) was extracted from six vestibular schwannoma tissue samples using the DNeasy Blood and Tissue Kit (Qiagen) following manufacturer's specifications. The concentration of double-stranded DNA in each sample was evaluated using a Qubit dsDNA BR Assay Kit. A minimum measurement of 50 ng/μl was required for each sample to be included with HaloPlex target enrichment.

Library Preparation and Targeted Capture

A library of DNA restriction fragments from all coding exons, introns, and UTRs (5′ and 3′) of the NF2 gene was prepared using a HaloPlex HS target enrichment kit (Agilent Technologies), following the manufacturer's instructions. The total region size was 95.045 kbp with an actual analyzed target of 89.408 kbp bases, which required 2581 amplicons to achieve this 94.07% coverage with maximum validation stringency. Enrichment was performed according to the supplier's protocol by the Ocular Genomics Institute at Massachusetts Eye and Ear (Boston, Mass., USA).

Briefly, 50 ng of genomic DNA from each sample diluted with nuclease-free water to a final concentration of 1.8 ng/L were digested in eight different reactions, each containing two restriction enzymes. Successful digestion of ECD gDNA was indicated by the appearance of three predominant bands at 124, 255, and 450 bp, corresponding to the 800-bp PCR product-derived restriction fragments. A library of HaloPlex probes designed using the HaloPlex SureDesign program (www.genomics.agilent.com) was hybridized to the library of genomic DNA restriction fragments. Enrichment was validated by gel electrophoresis. Following purification, the DNA concentration of each library was quantified using the high-sensitivity D1000 DNA Tapescreen analysis assay on the Tapestation 2200 instrument (Agilent Technologies), and samples were subsequently sequenced.

NF2 Gene Sequencing

Targeted enrichment sample sequencing was performed on an Illumina MiSeq NGS platform (Illumina, Inc.) by the Next Generation Sequencing Core of the Massachusetts Eye and Ear Ocular Genomics Institute. The purified and individually tagged amplicon libraries for each sample were pooled equimolarly, and a percentage of an internal control (ECD) was added to validate the DNA sequencing and to help balance the overall lack of sequence diversity. The sample pool was then placed in a MiSeq Reagent kit version 2 500-cycle cartridge (Illumina) containing sequencing reagents, and sequencing was performed on the Illumina MiSeq instrument by using a MiSeq Reagent Kit v2 flow cell (Illumina). The quality criteria for MiSeq includes a number of generated clusters between 600 and 1200 K/mm², >90% passed filter clusters, and approximately 5% sequenced ECD. To be included in the analysis, bases had at least a quality score of 40, and depth of coverage was at least 100 for all samples.

Bioinformatic Processing and Variant Prioritization

Raw data were demultiplexed and converted to fastq using Illumina bcl2fastq conversion software (v 2.16.0.10) as directed Agilent and Illumina. Prior to alignment, Agilent AGeNT (v3.5.1.46) was used to trim low-quality bases from the ends, remove adaptor sequences, and remove duplicated reads based on Molecular Barcode information following Agilent directions. Alignment was done by BWA (Burrows-Wheeler Aligner v0.7.13) “mem” algorithm using UCSC hg19 Human Reference Genome, variants and indels were called using GATK (Genome Analysis Toolkit v3.5) following the best practices, choosing HaplotypeCaller to generate a joint called Variant Call Format (VCF) file for all samples.

Genomic variant annotation was performed using ANNOVAR (ANNOtate VARiation v2016-02-01). A filter was applied to eliminate common variants as reported in the 1000 Genomes database. Data were visualized using the Integrative Genomics Viewer (IGV; Broad Institute, Cambridge, Mass.), and used to identify rare variants. To confirm accuracy of the sequencing read for rare variants, individual sample BAM files were visualized in the IGV software and analyzed for potential errors in sequencing. Using the 2017 release of the gnomAD browser (Broad Institute, Cambridge, Mass.), which contains exome sequence data from 123,136 individuals and whole genome sequencing from 15,496 individuals, remaining filtered variants were probed for previous reports in the literature. The Single Nucleotide Polymorphism database (dbSNP) was also referenced to determine whether rare variants identified by the gnomAD and 1000 Genomes databases were either novel or previously reported using this public-domain archive.

Statistical Methods

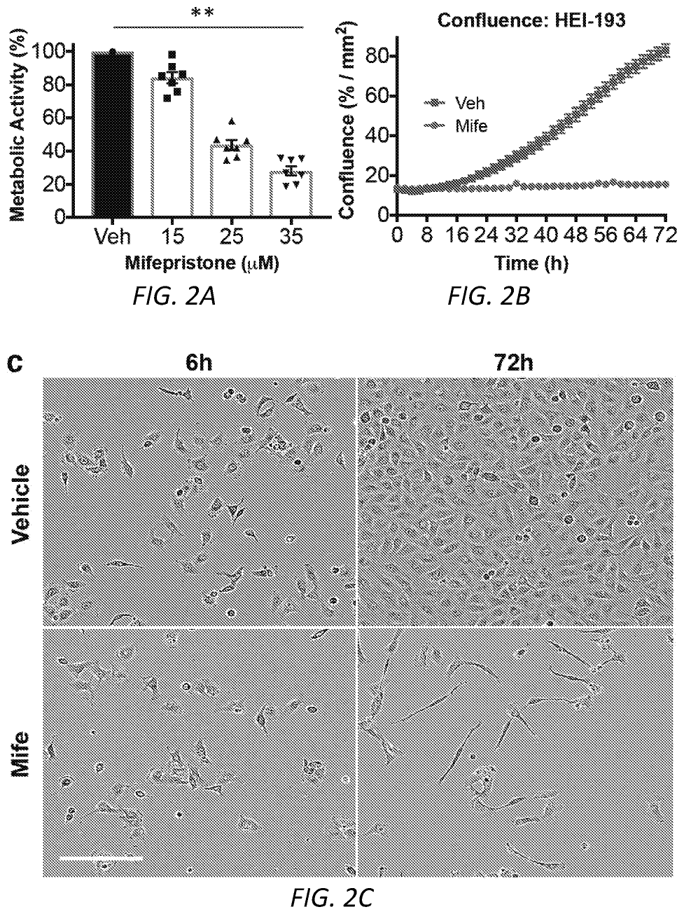

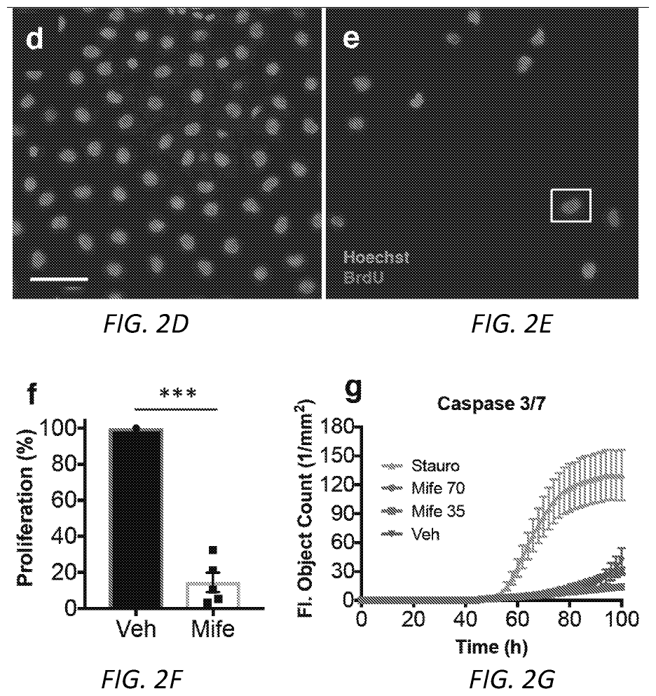

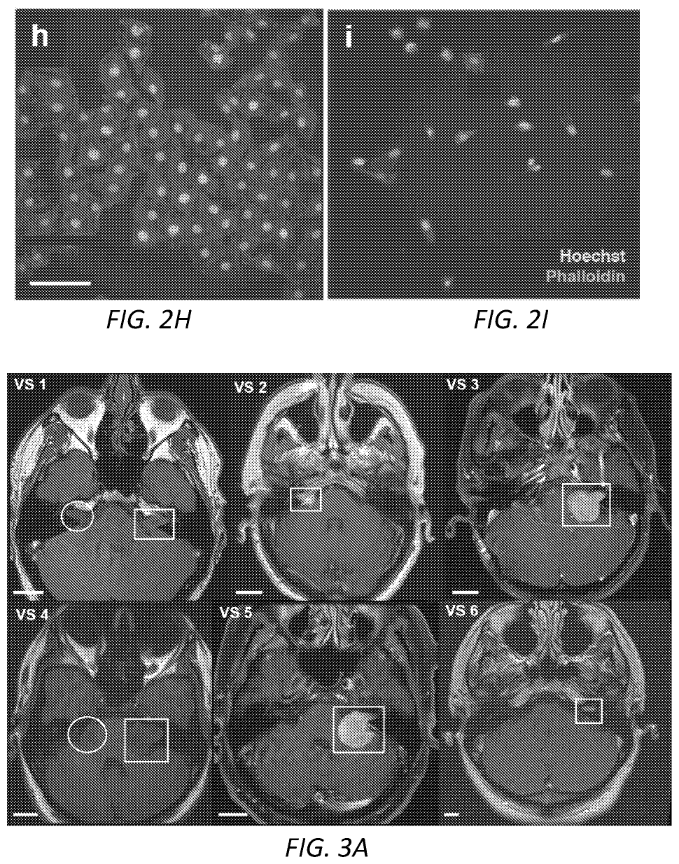

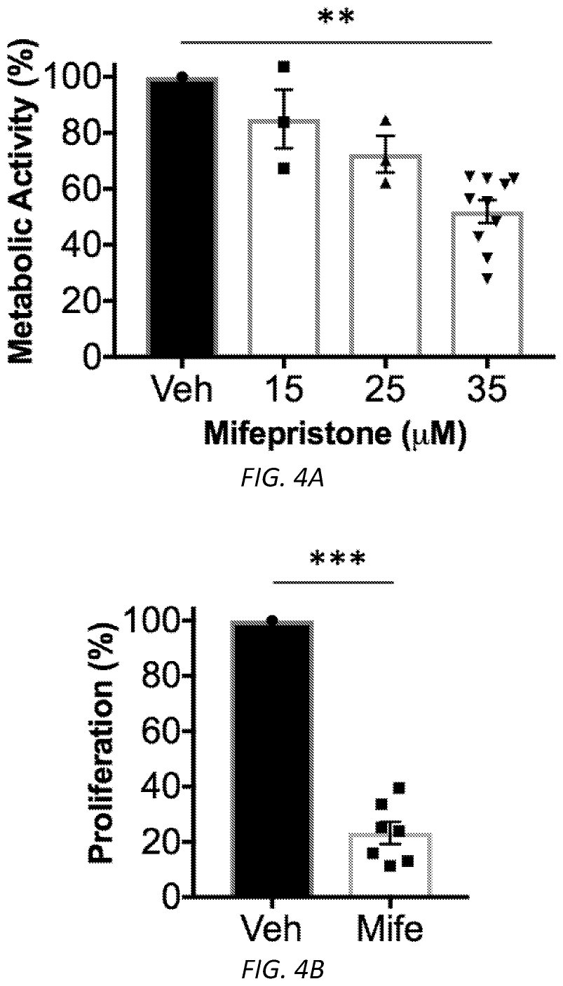

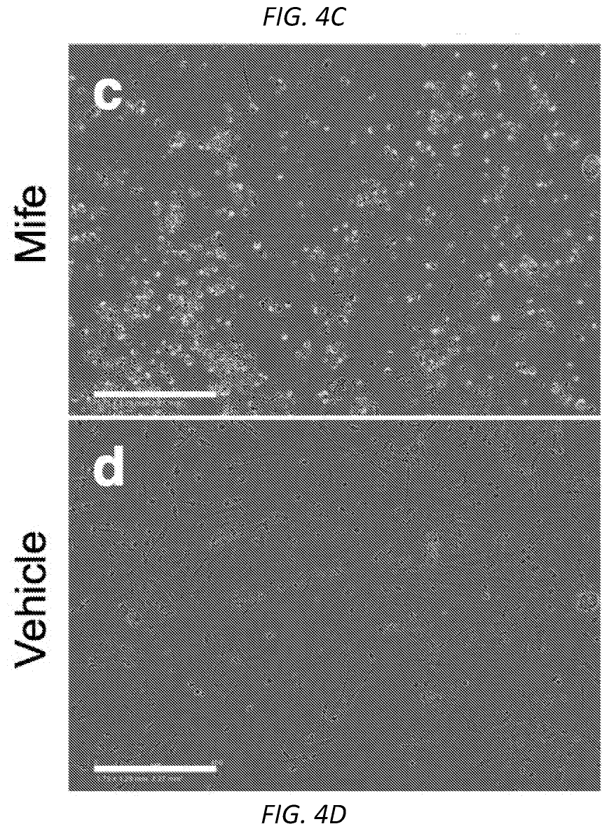

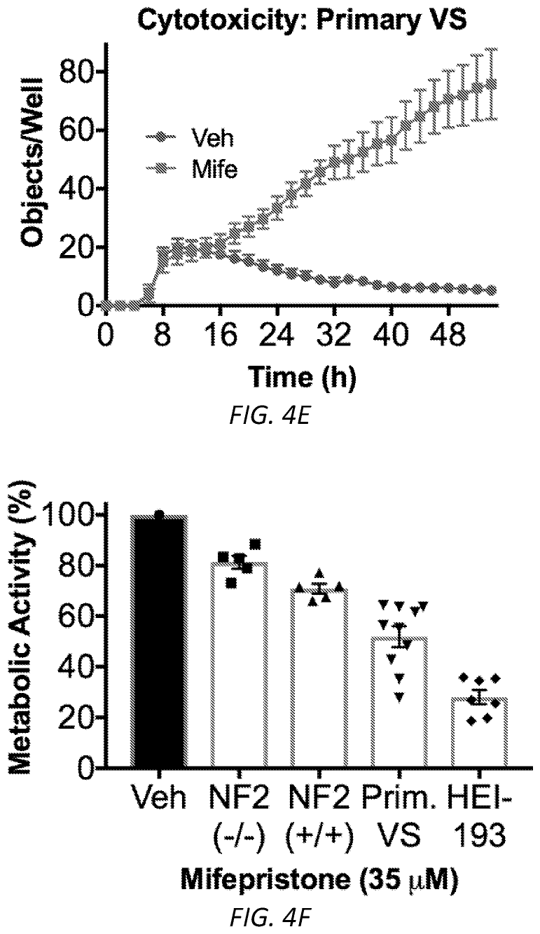

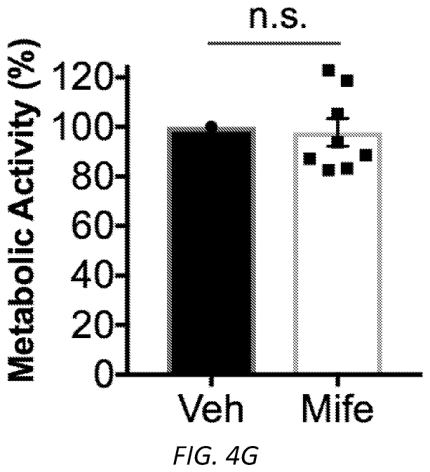

Throughout this paper, though figures present metabolic activity and cellular proliferation data as percentage of vehicle-treated control, statistical analyses were performed on raw data, following good statistical practice in pharmacology⁵³. Specifically, in FIG. 2a, FIG. 2f, FIG. 4a-b, and FIG. 4g, raw vehicle-treated and mifepristone-treated cell data are compared using two-way (“randomized block”) ANOVA, selected to minimize within-experiment variation by “blocking” treatment data with control data while meeting the equal-variance assumptions required by ANOVA⁵³. In FIG. 2a, comparison between control group and mifepristone-treated group (35 μM) was conducted using randomized block ANOVA on mean optical density values per treatment condition measured in eight independent experiments (p=0.006, F=42.46, DF=1). For FIG. 2f, randomized block ANOVA was performed between the ratio of BrdU+ to Hoescht+ cells per treatment condition in five independent experiments (p=0.0007, F=88.25, DF=1). For FIG. 4a, comparison between control group and mifepristone-treated group (35 μM) was conducted using randomized block ANOVA on mean optical density values per treatment condition measured in eight independent experiments (p=0.002, F=23.02, DF=1). In FIG. 4b, randomized block ANOVA was performed between the ratio of BrdU+ to Hoescht+ cells per treatment condition in seven independent experiments (p=0.0002, F=68.47, DF=1). In FIG. 4g, randomized block ANOVA was performed on mean optical density values per treatment condition measured in eight independent experiments (p=0.230, F=1.255, DF=1).

### Example 1. Preclinical Validation of Mifepristone for Vestibular Schwannoma

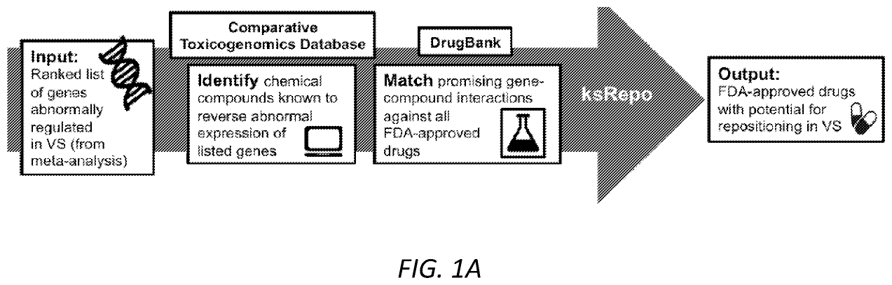

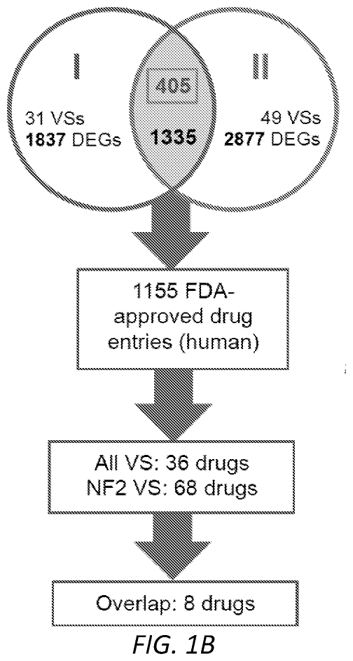

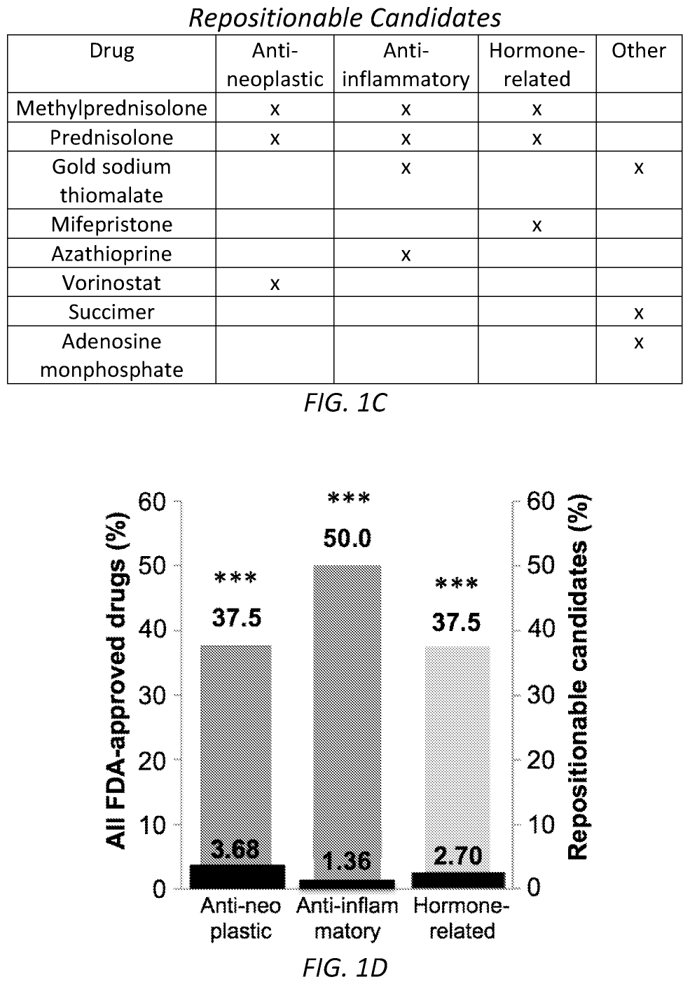

To identify FDA-approved drugs with potential for repositioning in VS, we conducted a computational screen using the open-source drug repositioning platform ksRepo, developed to screen expression profiles from any microarray or sequencing platform against any available database of gene-drug interactions³. ksRepo uses a modified Kolmogorov-Smimov statistic to compare a ranked list of differentially expressed genes (DEGs) characteristic of a given disease with transcriptional signatures associated with drugs known to interact with those genes, as publicly stored in the Comparative Toxicogenomics Database¹¹. From that list of drugs, ksRepo selects for compounds with entries in DrugBank, a compendium of FDA-approved drugs. The output is a list of FDA-approved drugs hypothesized to modulate genes with aberrant expression patterns in patients with disease (FIG. 1A). This approach was recently shown to be successful against a meta-analysis of DEGs from five independent prostate cancer datasets, from which ksRepo successfully predicted significance for five approved therapies in prostate cancer treatment³.

To provide robust input to ksRepo, we conducted the largest meta-analysis of primary human VS tissue to date, comprising genome-wide transcriptional microarray data from 80 tumors and 16 control nerves (FIG. 1B). Combined analysis of expression data from two large published datasets, one publicly available (NCBI GEO, GSE39645) and one donated by a collaborator⁶, yields 1,335 genes found to be commonly and concordantly dysregulated in VS, with 405 reaching significance after Bonferroni correction for multiple hypothesis testing (p<0.05). ksRepo takes the entire meta-analytic expression profile as input, not just that of significant genes, in order to screen a comprehensive picture of tumor-related gene expression against the known interactions of 1,155 FDA-approved drugs. As 13 of 80 VSs in the meta-analysis were harvested from patients with NF2, we also conducted a separate, parallel analysis comprising only NF2-associated tumors. ksRepo returned 36 drugs with potential for repositioning from the complete VS meta-analysis and 68 drugs from the NF2-specific analysis (Tables 1-2). Eight drugs appeared in both analyses and were prioritized for preclinical validation (FIG. 1C). Out of all FDA-approved drugs, this group of eight demonstrates significant enrichment for anti-inflammatory drugs (27.7-fold), hormone-related compounds (13.9-fold), and anti-neoplastic agents (13.6-fold) (FIG. 1D).

TABLE 1

| | | | | Bonferroni- |
|---|---|---|---|---|
| | Interacting | | Bootstrapped | Corrected |
| Compound Name | Genes | KS Score | P-Value | P-Value |
| ACETAMINOPHEN | 4257 | 0.028457 | 9.00E-04 | 0.038886 |
| ADENOSINE | | | | |
| MONOPHOSPHATE, | 609 | 0.08029 | 4.00E−04 | 0.020048 |
| ADENOSINE | | | | |
| POTASSIUM | 2232 | 0.046816 | 2.00E−04 | 0.011391 |
| ARSENIC TRIOXIDE | 2397 | 0.042501 | 0 | 0 |
| AZATHIOPRINE | 127 | 0.224742 | 0 | 0 |
| BELINOSTAT | 387 | 0.093239 | 0.001 | 0.041767 |
| ESTRADIOL | 4030 | 0.052971 | 0 | 0 |
| CARBAMAZEPINE | 1859 | 0.041777 | 0.0013 | 0.045247 |
| CYCLOPHOSPHAMIDE | 258 | 0.112747 | 9.00E−04 | 0.038886 |
| CYTARABINE | 541 | 0.13022 | 0 | 0 |
| DECITABINE | 1657 | 0.07517 | 0 | 0 |
| DIAZEPAM | 30 | 0.363707 | 0 | 0 |
| CISPLATIN | 2198 | 0.058084 | 0 | 0 |
| CALCITRIOL | 2217 | 0.057748 | 0 | 0 |
| FLUORODEOXYGLUCOSE | 1 | 0.999469 | 6.00E−04 | 0.027844 |
| GOLD SODIUM | | | | |
| THIOMALATE | 82 | 0.315293 | 0 | 0 |
| LOMEFLOXACIN | 1 | 0.999823 | 4.00E−04 | 0.020048 |
| METHYLPREDNISOLONE | 92 | 0.271815 | 0 | 0 |
| MIFEPRISTONE | 306 | 0.120264 | 0 | 0 |
| NALBUPHINE | 2 | 0.965334 | 0.0012 | 0.044224 |
| NORGESTIMATE | 19 | 0.422505 | 0.0012 | 0.044224 |
| LEVONORGESTREL | 185 | 0.152175 | 3.00E−04 | 0.016343 |
| OXYMETAZOLINE | 8 | 0.683056 | 1.00E−04 | 0.005967 |
| PANOBINOSTAT | 1042 | 0.11206 | 0 | 0 |
| PEROSPIRONE | 2 | 0.965334 | 0.0012 | 0.044224 |
| PREDNISOLONE | 119 | 0.210251 | 0 | 0 |
| PROGESTERONE | 1676 | 0.089375 | 0 | 0 |
| RALOXIFENE | 635 | 0.083944 | 0 | 0 |
| SUCCIMER | 260 | 0.111254 | 0.0013 | 0.045247 |
| TAMOXIFEN | 762 | 0.073528 | 0 | 0 |
| TRETINOIN | 3840 | 0.074244 | 0 | 0 |
| TROPICAMIDE | 7 | 0.637071 | 0.0012 | 0.044224 |
| VALPROIC ACID | 9378 | 0.038604 | 0 | 0 |
| VORINOSTAT | 871 | 0.106113 | 0 | 0 |
| XYLOMETAZOLINE | 7 | 0.683056 | 5.00E−04 | 0.024096 |
| ZOLEDRONIC ACID | 1318 | 0.076358 | 0 | 0 |

KS Score, Kolmogorov-Smirnov score.

TABLE 2

| | | | | Bonferroni- |
|---|---|---|---|---|
| | Interacting | | Bootstrapped | Corrected |
| Compound Name | Genes | KS Score | P-Value | P-Value |
| GLUTATHIONE | 144 | 0.147931 | 0.0014 | 0.031325 |
| ACETAMINOPHEN | 4257 | 0.037806 | 0 | 0 |
| ACETYLCYSTEINE | 344 | 0.105493 | 6.00E−04 | 0.017086 |
| ADEFOVIR DIPIVOXIL, | 96 | 0.189062 | 9.00E−04 | 0.022554 |
| ADEFOVIR | | | | |
| ADENOSINE | 269 | 0.102338 | 0.0023 | 0.043013 |
| ADENOSINE | | | | |
| MONOPHOSPHATE, | | | | |
| ADENOSINE | 609 | 0.073664 | 0.0015 | 0.032405 |
| ZINC | 1817 | 0.05048 | 1.00E−04 | 0.004321 |
| ALUMINUM | 133 | 0.146545 | 0.0026 | 0.047909 |
| AMOXICILLIN | 5 | 0.817003 | 3.00E−04 | 0.01074 |
| POTASSIUM | 2232 | 0.051543 | 0 | 0 |
| ARSENIC TRIOXIDE | 2397 | 0.058774 | 0 | 0 |
| AZATHIOPRINE | 127 | 0.172315 | 4.00E−04 | 0.012851 |
| BENZOYL PEROXIDE | 9 | 0.671167 | 1.00E−04 | 0.004321 |
| ESTRADIOL | 4030 | 0.039261 | 0 | 0 |
| SIMVASTATIN | 345 | 0.102268 | 7.00E−04 | 0.018662 |
| HEPARIN | 76 | 0.201638 | 0.0021 | 0.040482 |
| CAPSAICIN | 190 | 0.132492 | 0.0018 | 0.036974 |
| CARBAMAZEPINE | 1859 | 0.068489 | 0 | 0 |
| CELECOXIB | 101 | 0.198875 | 8.00E−04 | 0.020883 |
| CIDOFOVIR | 251 | 0.137126 | 1.00E−04 | 0.004321 |
| COPPER | 6405 | 0.027635 | 0 | 0 |
| CUPRIC CHLORIDE | 180 | 0.1683 | 0 | 0 |
| CYCLOSPORINE | 6697 | 0.046 | 0 | 0 |
| DAUNORUBICIN | 152 | 0.157382 | 3.00E−04 | 0.01074 |
| CISPLATIN | 2198 | 0.044117 | 5.00E−04 | 0.015663 |
| CALCITRIOL | 2217 | 0.056323 | 0 | 0 |
| DISULFIRAM | 505 | 0.095812 | 0 | 0 |
| DOXORUBICIN | 796 | 0.082771 | 0 | 0 |
| ETOPOSIDE | 439 | 0.085127 | 0.0018 | 0.036974 |
| EUGENOL | 151 | 0.146556 | 0.0013 | 0.029616 |
| FLUOROURACIL | 1100 | 0.056576 | 6.00E−04 | 0.017086 |
| FORMALDEHYDE | 2841 | 0.054947 | 0 | 0 |
| GADODIAMIDE | 39 | 0.275303 | 0.002 | 0.039778 |
| GEFITINIB | 92 | 0.203197 | 6.00E−04 | 0.017086 |
| GEMCITABINE | 228 | 0.148014 | 0 | 0 |
| GOLD SODIUM | | | | |
| THIOMALATE | 82 | 0.268746 | 0 | 0 |
| IFOSFAMIDE | 204 | 0.163148 | 1.00E−04 | 0.004321 |
| INDOMETHACIN | 304 | 0.111327 | 0.001 | 0.023642 |
| IRINOTECAN | 508 | 0.091101 | 2.00E−04 | 0.008084 |
| IRON | 172 | 0.132327 | 0.002 | 0.039778 |
| METHOXSALEN | 23 | 0.367622 | 9.00E−04 | 0.022554 |
| METHYLPREDNISOLONE | 92 | 0.239755 | 0 | 0 |
| MIFEPRISTONE | 306 | 0.123169 | 0 | 0 |
| NIMESULIDE | 70 | 0.231942 | 2.00E−04 | 0.008084 |
| NITROPRUSSIDE | 57 | 0.243079 | 0.001 | 0.023642 |
| OLEIC ACID | 41 | 0.275681 | 0.0021 | 0.040482 |
| OXYGEN | 1465 | 0.051363 | 1.00E−04 | 0.004321 |
| PACLITAXEL | 539 | 0.082731 | 4.00E−04 | 0.012851 |
| PENICILLAMINE | 48 | 0.272514 | 0.001 | 0.023642 |
| PF-2341066 | 1 | 0.998939 | 0.0011 | 0.025524 |
| PIROXICAM | 562 | 0.078578 | 7.00E−04 | 0.018662 |
| PREDNISOLONE | 119 | 0.168008 | 0.0015 | 0.032405 |
| PROGESTERONE | 1676 | 0.062716 | 0 | 0 |
| ISOTRETINOIN | 695 | 0.07242 | 7.00E−04 | 0.018662 |
| SUCCIMER | 260 | 0.136878 | 0 | 0 |
| SULFAPYRIDINE | 7 | 0.648441 | 4.00E−04 | 0.012851 |
| SURAMIN | 22 | 0.361042 | 0.0023 | 0.043013 |
| TESTOSTERONE | 1713 | 0.048875 | 3.00E−04 | 0.01074 |
| THALIDOMIDE | 133 | 0.163575 | 6.00E−04 | 0.017086 |
| TRETINOIN | 3840 | 0.042788 | 0 | 0 |
| VALPROIC ACID | 9378 | 0.035833 | 0 | 0 |
| VANCOMYCIN | 5 | 0.800731 | 3.00E−04 | 0.01074 |
| VINBLASTINE | 201 | 0.125458 | 0.0018 | 0.036974 |
| VINCRISTINE | 1026 | 0.08311 | 0 | 0 |
| RIBOFLAVIN | 18 | 0.456353 | 4.00E−04 | 0.012851 |
| VITAMIN E | 1354 | 0.074731 | 0 | 0 |
| VORINOSTAT | 871 | 0.08358 | 0 | 0 |
| ZIDOVUDINE | 346 | 0.140658 | 0 | 0 |

KS Score, Kolmogorov-Smirnov score.

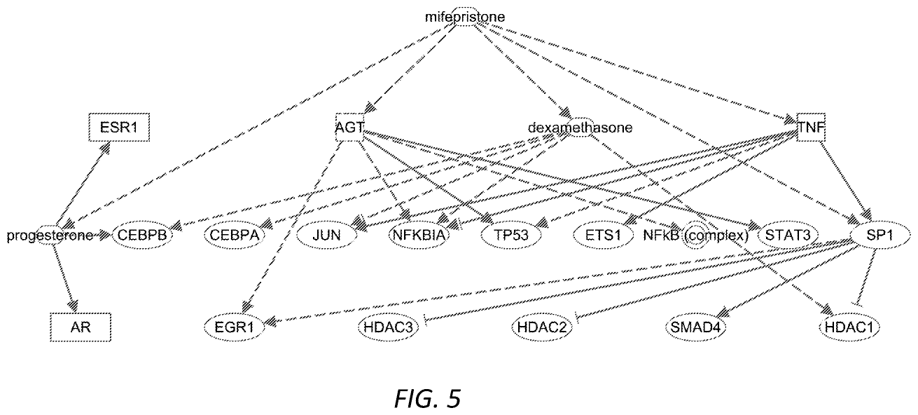

Independently of our computational repositioning analysis, when gene expression data from our meta-analysis was input to Ingenuity Pathway Analysis (Qiagen), mifepristone was predicted as a significant upstream regulator of the resulting networks (p=4.26*10⁻⁵) and theorized to act upstream of inflammatory markers characteristic of VS, such as TNF and NFkB^{30,31} (FIG. 5).

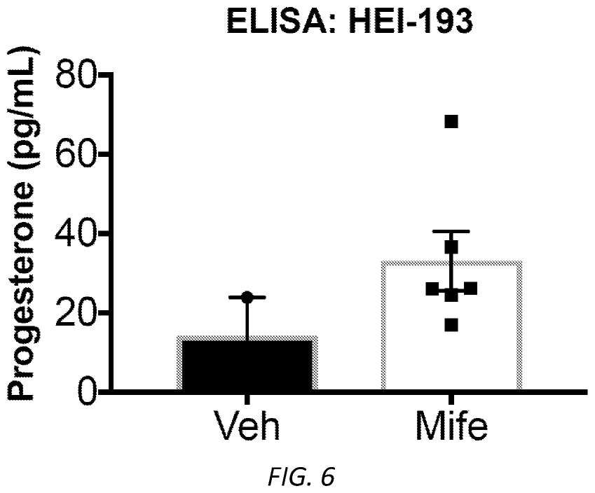

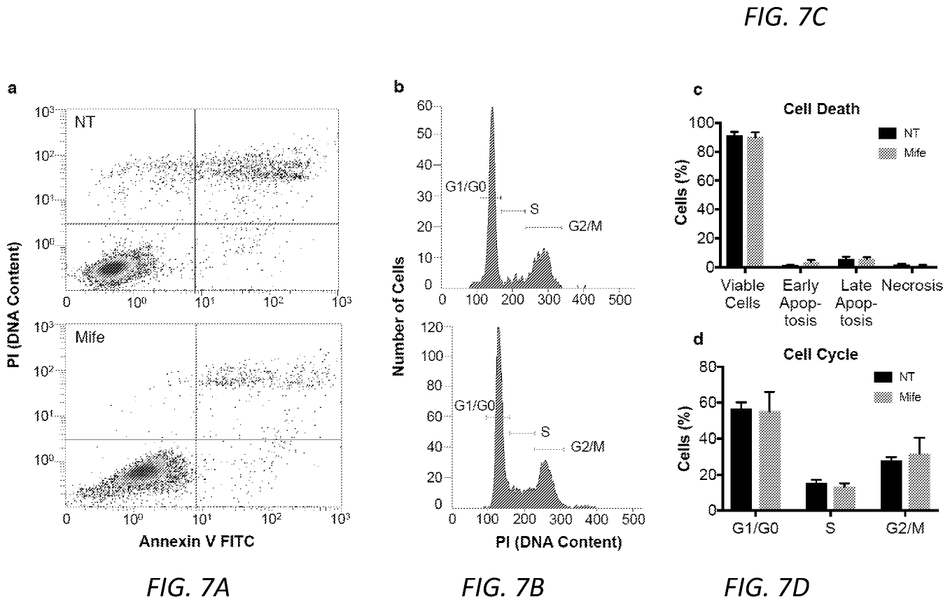

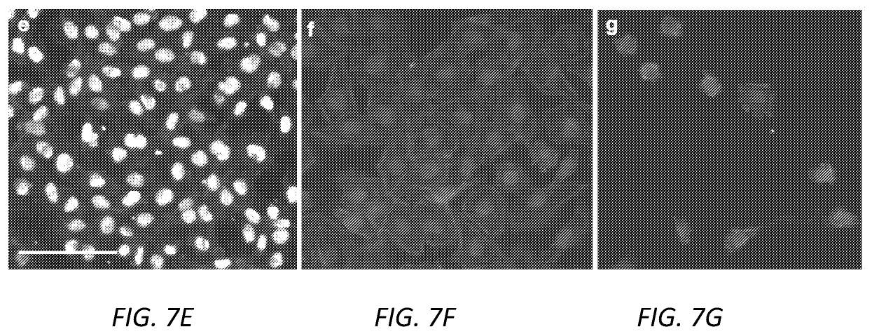

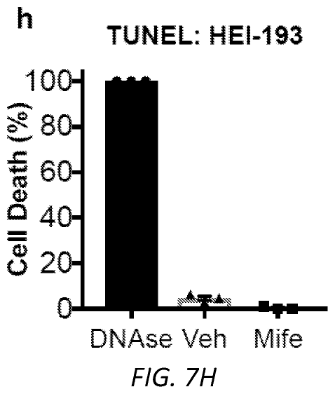

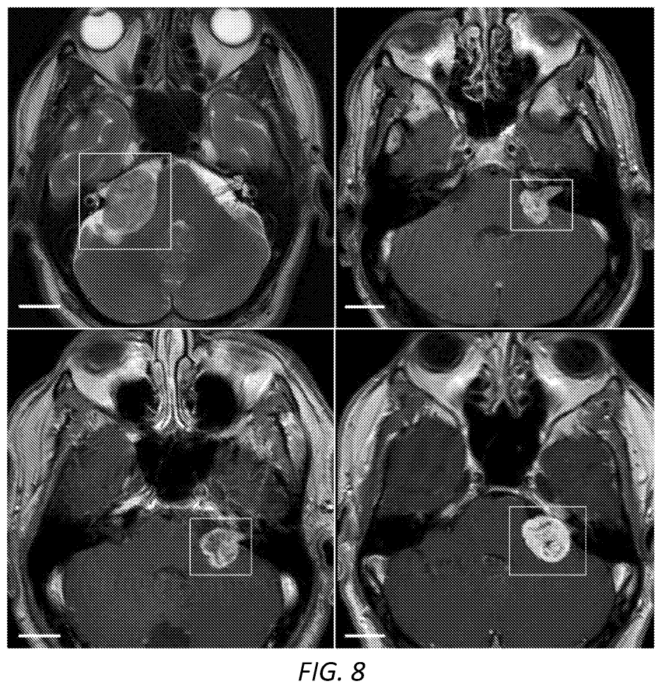

Administration of mifepristone to HEI-193 immortalized human schwannoma cells in culture produces a significant, dose-dependent response in metabolic activity (FIG. 2A); a significant reduction in cell confluence (FIG. 2B-C); and a dramatic decline in cellular proliferation (FIG. 2D-F). Mifepristone treatment does not produce a significant increase in the apoptotic or necrotic cell fraction among HEI-193 cells, suggesting that this drug may act by slowing proliferation rather than inducing cell death (FIG. 2G, FIGS. 7A-7H). Under mifepristone treatment, HEI-193 cells assume a long, thin, spindle-like shape (FIG. 2C). This observation is reported in previous studies of mifepristone treatment of ovarian, breast, prostate, and nerve cells, where such morphological changes are attributed to dysregulated distribution of f-actin and tubulin proteins in the cytoskeleton³². Continuous, dynamic actin remodeling is characteristic of NF2-deficient schwannoma cells³³, as the NF2 protein product, merlin, is known to selectively bind f-actin³⁴. Phalloidin staining confirms the shrunken, crumpled appearance of f-actin in mifepristone-treated cells (FIG. 2H-I). Enzyme-linked immunosorbent assay (ELISA) for progesterone in conditioned cell culture medium reveals higher levels of progesterone in medium collected from mifepristone-treated cells than from untreated cells, suggesting that mifepristone effectively competes with progesterone for receptor binding (FIG. 6).

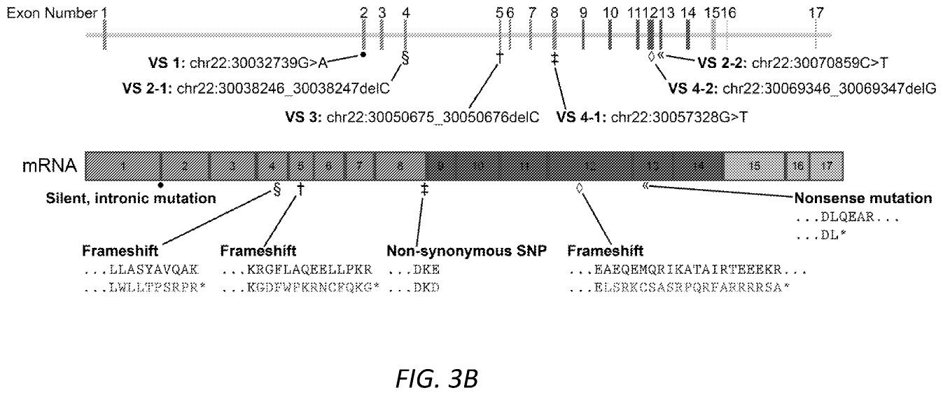

Current, large-scale meta-analyses of drug toxicology, bioavailability, and efficacy in animal models reveal a shocking lack of predictive power when compared to human data^{35,36}. Accordingly, the U.S. National Research Council has recommended the substitution of model animal testing with in vitro human cell-based assays and in silico modeling of diseases and networks³⁷. We evaluated the effect of mifepristone applied directly to primary human VS cells. Fresh VS tissue samples from eight human patients undergoing tumor resection surgery were collected and schwannoma cells grown in the laboratory according to our published protocols³⁸. Single-gene sequencing (Illumina MiSeq) of six treated VSs (FIG. 3A) revealed that four of six exhibit novel mutations in the NF2 gene, a fraction consistent with published literature⁷ (FIG. 3B). When applied to primary human VS cells, mifepristone produced a dose-dependent response in metabolic activity and a dramatic reduction in cellular proliferation (FIG. 4A-B). Live-cell fluorescence imaging revealed a marked increase in cytotoxicity in primary cultures (FIG. 4C-E). No correlation in drug response with tumor size or NF2 mutation type was observed.

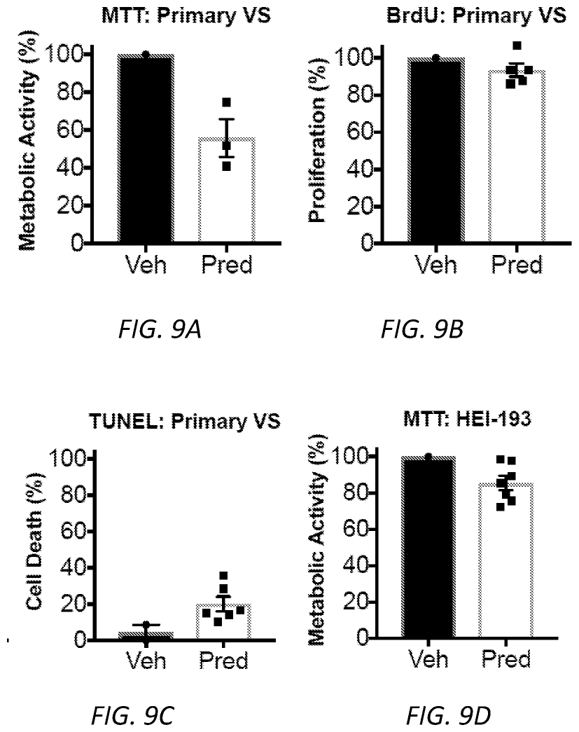

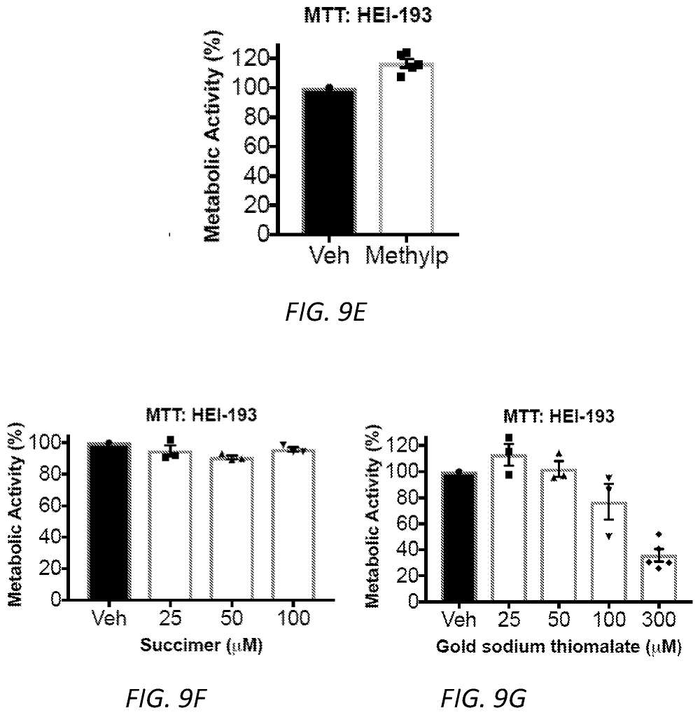

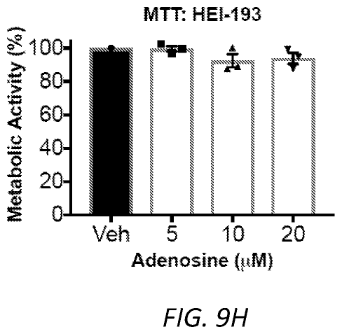

In a long-term clinical trial of mifepristone for unresectable meningioma, minor responses resulting in clinical benefit were noted in eight of 28 patients¹³, though a subsequent double-blind, randomized Phase III trial of 164 patients reported no difference between treatment and placebo³⁹. To evaluate the effect of this drug on schwannoma cells in comparison to meningioma cells, we compared mifepristone-treated primary human VS and HEI-193 cells to immortalized human arachnoid cells in which the NF2 gene has been excised by CRISPR⁴⁰. Primary VS cells responded more dramatically to mifepristone than human arachnoid cells with or without the NF2 gene (FIG. 4F), suggesting that schwannoma cells are more responsive than meningioma cells to this drug. Additionally, to ensure that mifepristone administration did not lead to adverse effects among healthy human Schwann cells, primary human Schwann cells were cultured from eight great auricular nerves harvested from patients undergoing benign parotidectomy or neck dissection³⁸. Treatment of these cells with mifepristone did not cause appreciable changes in metabolic activity (FIG. 4G). Preliminary testing of clinically reasonable concentrations of other drugs recommended by ksRepo, including adenosine monophosphate, gold sodium thiomalate, succimer, and methylprednisolone showed no effect on the metabolic activity of HEI-193 cells, though prednisolone produced modest effects when applied to primary human VS cells (FIG. 9A-9H).

The in silico repositioning of mifepristone for human VS using pooled human transcriptomic data, as well as the preclinical validation of this drug on primary human cells, constitutes a powerful case for the computational identification of novel indications for FDA-approved drugs. Mifepristone is safe and approved for human use and deserving of further attention for repurposing in a debilitating disease with no FDA-approved drug therapy.

### REFERENCES

1. Hay, M., Thomas, D. W., Craighead, J. L., Economides, C. & Rosenthal, J. Clinical development success rates for investigational drugs. Nat Biotechnol. 32, 40-51 (2014).
2. Li, J., et al. A survey of current trends in computational drug repositioning. Brief Bioinformatics. 17, 2-12 (2016).
3. Brown, A. S., Kong, S. W., Kohane, I. S. & Patel, C. J. ksRepo: a generalized platform for computational drug repositioning. BMC Bioinformatics. 17, 78 (2016).
4. Coelho, E. D., Arrais, J. P. & Oliveira, J. L. Computational discovery of putative leads for drug repositioning through drug-target interaction prediction. PLoS Comput Biol. 12, e1005219 (2016).
5. Gayvert, K. M., et al. A computational drug repositioning approach for targeting oncogenic transcription factors. Cell Rep. 15, 2348-56 (2016).
6. Agnihotri, S., et al. Gene-expression profiling elucidates molecular signaling networks that can be therapeutically targeted in vestibular schwannoma. J Neurosurg. 121, 434-45 (2014).
7. Hadfield, K. D., et al. Rates of loss of heterozygosity and mitotic recombination in NF2 schwannomas, sporadic vestibular schwannomas and schwannomatosis schwannomas. Oncogene. 29, 6216-21 (2010).
8. Asthagiri, A. R., et al. Mechanisms of hearing loss in neurofibromatosis type 2. PLoS ONE. 7, e46132 (2012).
9. Dilwali, S., Landegger, L. D., Soares, V. Y. R., Deschler, D. G. & Stankovic, K. M. Secreted factors from human vestibular schwannomas can cause cochlear damage. Sci Rep. 22,18599 (2015).
10. Plotkin, S. R., Merker, V. L., Muzikansky, A., Barker, F. G. & Slattery, W. Natural history of vestibular schwannoma growth and hearing decline in newly diagnosed neurofibromatosis type 2 patients. Otol Neurotol. 35, 50 (2014).
11. Davis, A. P., et al. The comparative toxicogenomics database's 10th year anniversary: update 2015. Nucleic Acids Res. 43, 914 (2015).
12. Check, J. H., Wilson, C., Cohen, R. & Sarumi, M. Evidence that mifepristone, a progesterone receptor antagonist, can cross the blood brain barrier and provide palliative benefits for glioblastoma multiforme grade IV. Anticancer Res. 34, 2385-8 (2014).
13. Grunberg, S. M., et al. Long-term administration of mifepristone (RU486): clinical tolerance during extended treatment of meningioma. Cancer Invest. 24, 727-33 (2006).
14. Touat, M., Lombardi, G., Farina, P., Kalamarides, M. & Sanson, M. Successful treatment of multiple intracranial meningiomas with the antiprogesterone receptor agent mifepristone (RU486). Acta Neurochir (Wien). 156, 1831-5 (2014).
15. Jurado, R., Lopez-Flores, A., Alvarez, A. & García-López, P. Cisplatin cytotoxicity is increased by mifepristone in cervical carcinoma: an in vitro and in vivo study. Oncol Rep. 22, 1237-45 (2009).
16. Segovia-Mendoza, M., et al. Antihormonal agents as a strategy to improve the effect of chemo-radiation in cervical cancer: in vitro and in vivo study. BMC Cancer. 15, 21 (2015).
17. Liu, R., et al. Mifepristone suppresses basal triple-negative breast cancer stem cells by down-regulating KLF5 expression. Theranostics. 6, 533-44 (2016).
18. Gaddy, V. T., et al. Mifepristone induces growth arrest, caspase activation, and apoptosis of estrogen receptor-expressing, antiestrogen-resistant breast cancer cells. Clin Cancer Res. 10, 5215-25 (2004).
19. Helmestam, M., Lindgren, K. E., Stavreus-Evers, A. & Olovsson, M. Mifepristone-exposured human endometrial endothelial cells in vitro. Reprod Sci. 21, 408-14 (2004).
20. Moe, B. T., Vereide, A. B., Orbo, A., Jaeger, R. & Sager, G. Levonorgestrel, medroxyprogesterone and progesterone cause a concentration-dependent reduction in endometrial cancer (Ishikawa) cell density, and high concentrations of progesterone and mifepristone act in synergy. Anticancer Res. 29, 1047-52 (2009).
21. Rose, F. V. & Barnea, E. R. Response of human ovarian carcinoma cell lines to antiprogestin mifepristone. Oncogene. 12, 999-1003 (1996).
22. Goyeneche, A. A., Car6n, R. W. & Telleria, C. M. Mifepristone inhibits ovarian cancer cell growth in vitro and in vivo. Clin Cancer Res. 13, 3370-9 (2007).
23. Li, D., et al. Effects of mifepristone on invasive and metastatic potential of human gastric adenocarcinoma cell line MKN-45 in vitro and in vivo. World J Gastroenterol. 10, 1726-9 (2004).
24. Sun, Q, et al. A study of mifepristone/IFN-γ-induced apoptosis of human cholangiocarcinoma cell line FRH-0201 in vitro. Onco Targets Ther. 5, 335-42 (2012).
25. El Etreby, M. F., Liang, Y., Johnson, M. H. & Lewis, R. W. Antitumor activity of mifepristone in the human LNCaP, LNCaP-C4, and LNCaP-C4-2 prostate cancer models in nude mice. Prostate. 42, 99-106 (2000).
26. Lin, M. F., Kawachi, M. H., Stallcup, M. R., Grunberg, S. M. & Lin, F. F. Growth inhibition of androgen-insensitive human prostate carcinoma cells by a 19-norsteroid derivative agent, mifepristone. Prostate. 26, 194-204 (1995).
27. Tieszen, C. R., Goyeneche, A. A., Brandhagen, B. N., Ortbahn, C. T. & Telleria, C. M. Antiprogestin mifepristone inhibits the growth of cancer cells of reproductive and non-reproductive origin regardless of progesterone receptor expression. BMC Cancer. 11, 207 (2011).
28. Check, J. H., Dix, E., Cohen, R., Check, D. & Wilson, C. Efficacy of the progesterone receptor antagonist mifepristone for palliative therapy of patients with a variety of advanced cancer types. Anticancer Res. 30, 623-8 (2010).
29. Check, J. H., Check, D., Cohen, R. & Sarumi, M. Mifepristone causing complete remission of rapidly advancing leukemia with measurement of progesterone-induced blocking factor. Anticancer Res. 34, 2413-6 (2014).
30. Dilwali, S., et al. Preclinical validation of anti-nuclear factor-kappa B therapy to inhibit human vestibular schwannoma growth. Mol Oncol. 9, 1359-70 (2015).
31. Dilwali, S., Kao, S., Fujita, T., Landegger, L. D. & Stankovic, K. M. Nonsteroidal anti-inflammatory medications are cytostatic against human vestibular schwannomas. Transl Res. 166, 1-11 (2015).
32. Brandhagen, B. N., et al. Cytostasis and morphological changes induced by mifepristone in human metastatic cancer cells involve cytoskeletal filamentous actin reorganization and impairment of cell adhesion dynamics. BMC Cancer. 13, 35 (2013).
33. Flaiz, C., Kaempchen, K., Matthies, C. & Hanemann, C. O. Actin-rich protrusions and nonlocalized GTPase activation in merlin-deficient schwannomas. J Neuropathol Exp Neurol. 66, 608-16 (2007).
34. James, M. F., Manchanda, N., Gonzalez-Agosti, C., Hartwig, J. H. & Ramesh, V. The neurofibromatosis 2 protein product merlin selectively binds f-actin but not g-actin, and stabilizes the filaments through a lateral association. Biochem J. 356, 377-86 (2001).
35. Denayer, T., Stöhr, T. & Van Roy, M. Animal models in translational medicine: Validation and prediction. New Horizons in Translational Medicine. 2, 5-11 (2014).
36. Shanks, N., Greek, R. & Greek, J. Are animal models predictive for humans? Philos Ethics Humanit Med. 4, 2 (2009).
37. Krewski, D., et al. Toxicity testing in the 21st century: a vision and a strategy. J Toxicol Environ Health B Crit Rev. 13, 51-138 (2010).
38. Dilwali, S., et al. Primary culture of human Schwann and schwannoma cells: improved and simplified protocol. Hear Res. 315, 25-33 (2014).
39. Ji, Y., et al. Double-blind phase III randomized trial of the antiprogestin agent mifepristone in the treatment of unresectable meningioma: SWOG S9005. J Clin Oncol. 33, 4093-8 (2015).
40. Beauchamp, R. L., et al. A high-throughput kinome screen reveals serum/glucocorticoid-regulated kinase 1 as a therapeutic target for NF2-deficient meningiomas. Oncotarget. 6,16981-97 (2015).
41. Torres-Martin, M., et al. Microarray analysis of gene expression in vestibular schwannomas reveals SPP1/MET signaling pathway and androgen receptor deregulation. Int J Oncol. 42, 848-62 (2013).
42. Barrett, T., et al. NCBI GEO: archive for functional genomics data sets—update. Nucleic Acids Res. 41, 991 (2013).
43. Ritchie, M. E., et al. limma powers differential expression analyses for RNA-sequencing and microarray studies. Nucleic Acids Res. 43, e47 (2015).
44. Development Core Team R. R: A language and environment for statistical computing. Vienna, Austria: the R Foundation for Statistical Computing (2011).
45. Friedman, H. Simplified determinations of statistical power, magnitude of effect and research sample sizes. Educ Psychol Meas (1982).
46. Lumley, T. rmeta: Meta-analysis. R Package Version 2009. 2 (2009).
47. Benjamini, Y. & Hochberg, Y. Controlling the false discovery rate: a practical and powerful approach to multiple testing. J R Stat Soc Series B Stat Methodol 57, 289-300 (1995).
48. Gautier, L., Cope, L., Bolstad, B. M. & Irizarry, R. A. affy—analysis of Affymetrix GeneChip data at the probe level. Bioinformatics. 20, 307-15 (2004).
49. Irizarry, R. A., et al. Exploration, normalization, and summaries of high density oligonucleotide array probe level data. Biostatistics. 4, 249-64 (2003).
50. Carlson, M. org.Hs.eg.db: Genome wide annotation for human. https://bioconductor.org/packages/release/data/annotation/html/org.hs.eg.db.html (2016).
51. Wishart, D. S., et al. DrugBank: a comprehensive resource for in silico drug discovery and exploration. Nucleic Acids Res. 34, 668 (2006).
52. Lepont, P., et al. Point mutation in the NF2 gene of HEI-193 human schwannoma cells results in the expression of a merlin isoform with attenuated growth suppressive activity. Mutat Res. 637, 142-51 (2008).
53. Lew, M. Good statistical practice in pharmacology: problem 2. Br J Pharmacol 152, 299-303 (2007).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing the proliferation of a vestibular schwannoma cell, wherein the method comprises contacting the vestibular schwannoma cell with an effective concentration of mifepristone.

2. A method of treating a subject having vestibular schwannoma, wherein the method comprises administering to the subject a therapeutically effective amount of mifepristone.

3. The method of claim 2, wherein treating comprises reducing the rate of vestibular schwannoma tumor growth in the subject that include administering to the subject a therapeutically effective amount of mifepristone.

4. The method of claim 1, wherein the vestibular schwannoma cell is ex vivo.

5. The method of claim 1, wherein the vestibular schwannoma cell is in a subject.

6. The method of claim 2, wherein the subject is a human.

7. The method of claim 2, wherein the subject has been diagnosed as having vestibular schwannoma.

8. The method of claim 2, wherein the mifepristone is administered orally at a dose of 200 mg/day.

9. The method of claim 2, wherein the administration is local administration.

10. The method of claim 9, wherein the local administration is injection through the ear drum.

11. The method of claim 9, wherein the local administration is direct delivery into the inner ear or into the vestibular schwannoma tumor.

12. The method of claim 2, wherein the administration is systemic administration.

13. The method of claim 12, wherein the systemic administration is oral.

14. The method of claim 2, wherein the subject has been diagnosed as having vestibular schwannoma.

15. The method of claim 14, further comprising diagnosing the subject as having vestibular schwannoma.

16. The method of claim 2, wherein the subject does not have, or has not been diagnosed with, a neurofibroma.

17. The method of claim 2, wherein the subject has, or has been diagnosed with, a multiple schwannoma disorder.

18. The method of claim 17, further comprising diagnosing the subject as having a multiple schwannoma disorder.

19. The method of claim 17, wherein the multiple schwannoma disorder is neurofibromatosis type 2, schwannomatosis, or Carney complex.

20. The method of claim 17, wherein the subject has bilateral vestibular schwannomas.

21-39. (canceled)
